(19) United States
(12) Patent Application Publication
(10) Pub. No.: US 2009/0036841 A1
(43) Pub. Date: Feb. 5, 2009

(54) COATINGS FOR BLOOD AND BONE MARROW-CONTACTING DEVICES

(76) Inventors: James M. McKale, Leesburg, IN (US); Barbara S. Gibbs, West Lafayette, IN (US)

Correspondence Address: BOSE MCKINNEY & EVANS LLP, 111 MONUMENT CIRCLE, SUITE 2700, INDIANAPOLIS, IN, 46204, US

(21) Appl. No.: 12/184,869

(22) Filed: Aug. 1, 2008

Related U.S. Application Data
(60) Provisional application No. 60/953,356, filed on Aug. 1, 2007.

(51) Int. Cl. A61M 5/32

(52) U.S. Cl. 604/266

(57) ABSTRACT

Medical devices having an anticoagulant coating are described. The devices may be needles, syringes and blood processing devices. The anticoagulant coating can be any of heparin, heparin salts, citric acid salts, ethylenediaminetetraacetic acid salts, hirudin, sodium pentosan polysulfate, cumarin, derivatives of cumarin, warfarin, or phenprocoumon acenocoumarolor.

This application claims priority to U.S. Provisional Patent Application Ser. No. 60/953,356 filed Aug. 1, 2007, incorporated herein by reference.

### BACKGROUND OF THE RELATED ART

Blood products such as plasma, platelet rich plasma and platelet poor plasma are commonly used for surgical and orthopedic procedures. Autologous blood products are preferred and are isolated from blood or bone marrow aspirate obtained from a patient. The desired blood products may then be isolated using a centrifugal device or apparatus. An anticoagulant such as heparin or ACD-A is usually added to the blood or bone marrow aspirate sample to prevent the blood from clotting before it can be separated into the desired components. If the isolated blood product is to be used in a gel or solid form, the anticoagulant must either be removed or neutralized. Therefore the amount of anticoagulant is just sufficient to keep the blood or bone marrow aspirate from clotting.

At these anticoagulant concentrations, clotting may still occur when the blood or bone marrow aspirate comes in contact with a surface of a needle, syringe or blood processing device. This clotting may affect either the yield and/or the quality of the isolated blood fractions.

Thus it would be desirable to have needles, syringes or other blood processing devices that would prevent any clotting resulting from contact of blood or bone marrow aspirate with a surface, particularly a plastic surface. It would be further desirable to have blood processing devices which would eliminate the need for direct addition of anticoagulants to blood or bone marrow aspirate. Eliminating the direct addition of anticoagulants would allow for easier clotting of the subsequent isolated blood fractions, if desired.

### SUMMARY OF THE INVENTION

The invention relates generally to anticoagulant coatings and more particularly to anticoagulant coatings such as heparin coated onto aspirate needles, syringes, and blood processing devices that process blood and bone marrow aspirate.

In one aspect of the present invention there is provided a blood processing device having an anticoagulation coating. The coating may be an anticoagulant such as, but not limited to heparin, or it may be a hydrophobic or charged molecule that neutralizes a charge on the surface that is coated. The coating may be covalently linked to the surface or it may adhere to the surface by non-covalent forces such as ionic interactions, hydrogen bonding or van der waals interactions.

In another aspect of the present invention, there is provided a bone marrow aspirate needle having an anticoagulation coating. The coating may be an anticoagulant such as, but not limited to heparin, or it may be a hydrophobic or charged molecule that neutralizes a charge on the surface that is coated. The coating may be covalently linked to the surface or it may adhere to the surface by non-covalent forces such as ionic interactions, hydrogen bonding or van der waals interactions.

In a further aspect of the present invention there is provided a syringe having an anticoagulant coating. The coating may be an anticoagulant such as, but not limited to heparin, or it may be a hydrophobic or charged molecule that neutralizes a charge on the surface that is coated. The coating may be covalently linked to the surface or it may adhere to the surface by non-covalent forces such as ionic interactions, hydrogen bonding or van der waals interactions.

In yet another aspect of the present invention there is provided a method of fractionating blood or bone marrow aspirate using a needle, syringe and or blood processing device wherein at least one of the needle, syringe or blood processing device comprises an anticoagulant coating.

These and other features, aspects and advantages of the present invention will become better understood with reference to the following drawings, description and claims.

### BRIEF DESCRIPTION OF THE DRAWINGS

The present teachings will become more fully understood from the detailed description and the accompanying drawings, wherein:

FIG. 1 is an environmental view of a fractionation device including a suspension fractionated during the centrifuge process;

FIG. 2 is an environmental view of a separation container and a buoy;

FIG. 3 is an environmental view of a bone marrow transplantation needle; and

FIG. 4 is an environmental view of a bone marrow transplantation needle with an attached syringe.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

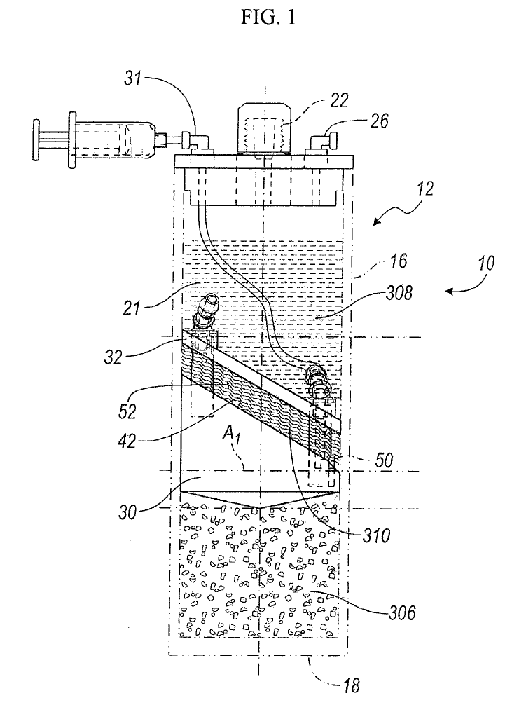

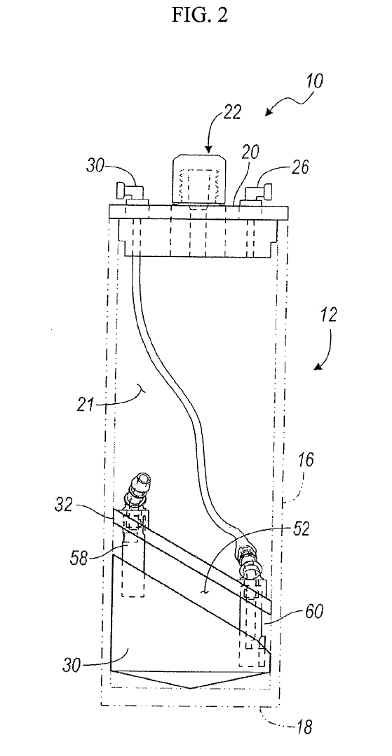

Broadly, the present invention provides devices comprising an anticoagulant coating on at least one surface of a device where that surface may come in contact with blood or bone marrow aspirate. The anticoagulant coating may comprise an anticoagulant such as, but not limited to heparin, or another compound that changes or neutralizes any charge on the surface. For convenience, cost and hygiene many devices are now made of plastic. Most plastics are made of polymers such as polyethylene that can carry a charge at the surface. The devices may comprise needles for collecting a sample, syringes or blood processing devices, particularly blood processing devices which separate blood into individual components such as plasma and red blood cells. A non-limiting example of such a device is disclosed in U.S. Patent Application Ser. No. 60/911,407 filed Apr. 12, 2007 (incorporated by reference herein) and illustrated in FIGS. 1 and 2. The unit is also available commercially under the brand name GPS.

The term “device” will be used broadly to define anything that may be used in collecting or processing blood or bone marrow aspirate including, but not limited to, needles, syringes and blood processing devices such as GPS or Vortech devices. The term “blood” will be used broadly to define blood, bone marrow aspirate or any blood related product or fluid. The term “coating” or “bonding” will be used broadly for adhering an anticoagulant to a blood contacting member. The term “blood contacting member” will be used broadly to include, but not limited to, syringes, needles, containers, buoys, and access ports, and the like.

In one embodiment, the coating comprises an anticoagulant such as, but not limited to, heparin and salts thereof, salts of citric acid, salts of ethylenediaminetetraacetic acid, hirudin, sodium pentosan polysulfate, cumarin and derivatives thereof, warfarin, phenprocoumon or acenocoumarol. The anticoagulants can be applied to the surface of the device as an aqueous solution or in an organic solvent. It may be applied by any of the techniques known in the art such as those disclosed in U.S. Pat. Nos. 6,626,874 and 4,808,499 (for needles) and U.S. patent application Ser. No. 10/293,978 (now U.S. Patent Publication No. 2003/0120198) (for syringes), herein incorporated by reference. In one illustrative embodiment, the anticoagulant is heparin. In a further illustrative embodiment, the heparin is coated onto the surface of the device by applying a solution of 1000 U/ml of heparin to the surface of the device and then removing an excess solution before a blood sample is placed in the device. In another illustrative embodiment, the heparin solution is lyophilized onto the surface of the device and the device stored for future use.

In another embodiment, the amount of anticoagulant may be from about 10 U/mm² to about 500 U/mm² on the surface of the device. In an illustrative embodiment, the amount of anticoagulant may be from about 40 U/mm² to about 200 U/mm² on the surface of the device. The amount of anticoagulant in the coating may depend on whether an additional anticoagulant in added to the blood sample. For example, if ACD-A (acid-citrate-dextrose) is added to the sample, the amount of anticoagulant in the coating may be less than if ACD-A is not added.

In an alternate embodiment of the invention, the coating comprises a hydrophobic or negatively charged compound. Platelets are negatively charged at physiological pH, and therefore coating the surface of a device with either a hydrophobic compound or a negatively charged compound would prevent the interaction of the platelets with the surface of the device, reducing or eliminating coagulation.

In one embodiment, the coatings of the present invention may be applied such that the compounds that comprise the coating are covalently attached to the surface. Linkers for covalently attaching compounds to plastic surfaces are well known in the art. In an alternate embodiment, the compounds may be incorporated or impregnated into the plastics themselves. In another alternate embodiment, the coatings interact with the surface of the device by non-covalent interactions such as ionic bonding, hydrogen bonding or through van der waals interactions. In a further embodiment, the surface may be pretreated before application of the anticoagulant coating wherein the pretreatment aids in the retention of the coating on the surface. In an illustrative embodiment, the surface is pretreated with an amphiphilic compound as disclosed in U.S. patent application Ser. No. 10/422,152 (now U.S. Patent Publication No. 2005/0037132), herein incorporated by reference.

In another embodiment, the present invention comprises an anticoagulant coating on at least one surface of a device where that surface may come in contact with blood or bone marrow aspirate. In yet another embodiment, the anticoagulant coating may be on at least one surface of a device where that surface may come in contact with platelets. In an illustrative embodiment, all surfaces of a device which come in contact with platelets are coated with the anticoagulant coating of the present invention.

In one embodiment, the device is a buoy suspension fractionation system such as, but not limited to, those illustrated in FIGS. 1 and 2. FIGS. 1 and 2 show a buoy suspension fractionation system 10, according to various embodiments that can be used in a clinical or laboratory environment to isolate fractions from a suspension or multi-component material removed from a patient. The multi-component material can include a sample of blood, bone marrow aspirate, adipose tissue, and the isolated fractions can include platelets, platelet poor plasma, platelet rich plasma and stromal cells. Isolated fractions can be used in a variety of clinical applications, animal applications, and laboratory applications. Some of the clinical applications include orthopedic surgery, plastic surgery, oral surgery, cardio-thoracic surgery, and wound healing. Animal applications can include equine, canine, etc. medicine. Laboratory applications include creating or synthesizing therapeutic materials from fractions produced by the fractionation system.

The suspension fractionation system 10 comprises a separation container 12 and a buoy 30. The separation container 12 can be a separation tube and having a container wall 16, a container bottom 18, a container top 20 enclosing a volume 21 that can be accessed by an access port 22, 26, 30. The separation container 12 can also have any appropriate shape, such as an oval provided, the buoy 30 is shaped to conform to the separation container 12. The separation container 12 can also have more than one compartment such as a separation tube and area to transfer tube contents such as platelet poor plasma away from the separation tube 12. When the separation container 12 is at rest, a buoy perimeter and the container wall 16 can form an interference fit to hold the buoy 30 at a position in the separation container 12. When the separation container 12 is centrifuged the buoy perimeter and the container wall 16 have clearance allowing the buoy to move within the separation container 12 and a material to pass between the buoy perimeter and the container wall. For example, the container 12 can compress axially to increase its internal diameter. Alternatively, the buoy 30 could have an opening, such as a central opening, that would allow a material to move through the buoy.

It is contemplated that the inner surface of the container 12 and the surfaces of the buoy 30 may be coated with the anticoagulant coating of the present invention. It is further contemplated that the access ports 22, 26, 30 and any tubing associated with the buoy suspension fractionation system 10 may also be coated with the anticoagulant coating of the present invention.

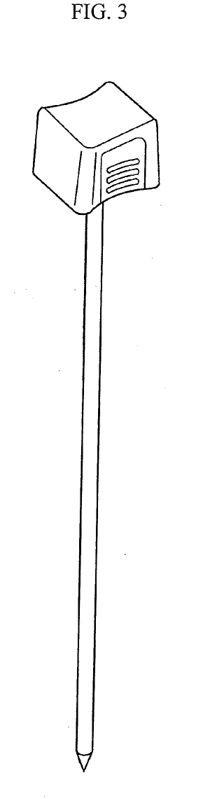

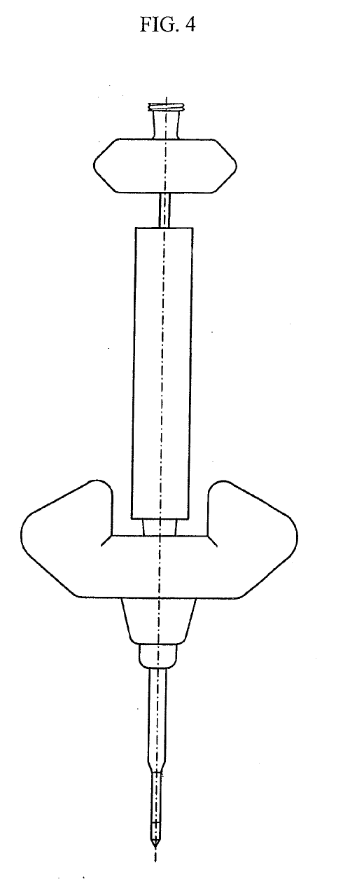

In yet another embodiment, a bone marrow transplantation needle, such as, but not limited to, those shown in FIGS. 3 and 4 may be coated with the anticoagulant coating of the present invention. It may be desirable to coat the inside of the needle as well as the hub that the needle is attached to. It may be further desirable to coat a syringe attached to the needle with the anticoagulant coatings of the present invention.

In a further embodiment, a kit is supplied having at least one device comprising the anticoagulant coating of the present invention.

It should be understood, of course, that the foregoing relates to exemplary embodiments of the invention and that modifications may be made without departing from the spirit and scope of the invention as set forth in the following claims.

What is claimed is:

1. A blood processing device, comprising:
a blood contacting member; and
an anticoagulant coated on the surface of or impregnated in the blood contacting member, whereby blood which contacts the blood contacting member is prevented from clotting.

2. The blood processing device of claim 1, wherein the anticoagulant is covalently adhered to the blood contacting member.

3. The blood processing device of claim 1, wherein the anticoagulant is non-covalent adhered to the blood contacting member.

4. The blood processing device of claim 3, wherein the anticoagulant is adhered to the blood contacting member by ionic interactions.

5. The blood processing device of claim 3, wherein the anticoagulant is adhered to the blood contacting member by hydrogen interactions.

6. The blood processing device of claim 3, wherein the anticoagulant is adhered to the blood contacting member by van der waals interactions.

7. The blood processing device of claim 1, wherein the anticoagulant is hydrophobic.

8. The blood processing device of claim 1, wherein the anticoagulant is negatively charged.

9. The blood processing device of claim 1, wherein the blood contacting member is pretreated with an anticoagulant retention aid prior to coating the blood contacting member.

10. The blood processing device of claim 9, wherein the anticoagulant retention aid pretreatment is amphiphilic.

11. The blood processing device of claim 1, wherein the anticoagulant comprises one or more of heparin, heparin salts, citric acid salts, ethylenediaminetetraacetic acid salts, hirudin, sodium pentosan polysulfate, cumarin, derivatives of cumarin, warfarin, or phenprocoumon acenocoumarolor.

12. The blood processing device of claim 1, wherein the anticoagulant is applied to the blood contacting member in an amount of from about 10 U/mm² to about 500 U/mm².

13. The blood processing device of claim 1, wherein the anticoagulant is applied to the blood contacting member in an amount of from about 40 U/mm² to about 200 U/mm².

14. The blood processing device of claim 1, wherein the anticoagulant is adhered to at least one surface of the blood contacting member, wherein the at least one surface interfaces with blood or bone marrow aspirate.

15. The blood processing device of claim 1, wherein the anticoagulant is adhered to at least one surface of the blood contacting member, wherein the at least one surface interfaces with platelets.

16. The blood processing device of claim 1, wherein the blood processing device is a syringe and the blood contacting member is a needle.

17. The blood processing device of claim 1, wherein the blood processing device is a bone marrow transplantation device and the blood contacting member is a needle.

18. The blood processing device of claim 1, wherein the blood processing device is a fractionation device and the blood contacting member comprises one or more of separation container, buoy, or access ports.
